(19) United States
(12) Patent Application Publication
(10) Pub. No.: US 2016/0002582 A1
(43) Pub. Date: Jan. 7, 2016

(54) High Efficiency Biometric Device For Producing Hydrogen And Methane

(71) Applicants: Chiu-Yue Lin, Taichung City (TW); Ping-Jei Lin, Taichung City (TW); Chyi-How Lay, Taichung City (TW); Chin-Chao Chen, Taichung City (TW); You-Chyuan Liang, Taichung City (TW); Huang-Chih Lu, Taichung City (TW); Chen-Yeon Chu, Taichung City (TW)

(72) Inventors: Chiu-Yue Lin, Taichung City (TW); Ping-Jei Lin, Taichung City (TW); Chyi-How Lay, Taichung City (TW); Chin-Chao Chen, Taichung City (TW); You-Chyuan Liang, Taichung City (TW); Huang-Chih Lu, Taichung City (TW); Chen-Yeon Chu, Taichung City (TW)

(21) Appl. No.: 14/324,253

(22) Filed: Jul. 7, 2014

(51) Int. Cl. C12M 1/107; C12M 1/00; C12M 1/02

(52) U.S. Cl. CPC C12M 21/04; C12M 27/00; C12M 47/10

(57) ABSTRACT

A high efficiency biometric device for producing hydrogen and methane mainly uses a two-stage anaerobic fermentation device to transform an organic wastewater mixed solution into hydrogen, methane, carbon dioxide and a removal liquid, and then uses a solid liquid separation tank to filter and separate the removal liquid to reduce the sludge and obtain the treated water with a good property. The characteristic lies in that the two-stage anaerobic fermentation device comprises a first anaerobic fermentation tank, a neutralization tank, a feeding tank and a second anaerobic fermentation tank composed of anaerobic fermentation tanks disposed in parallel. The biometric device can increase the removal rate and the methane recovery, and also has a generator for combusting the recycled gas to advantageously have the low energy consumption. Thus, the organic waste contamination is decreased, and the green energy production pathway is also increased.

### BACKGROUND OF THE INVENTION

(1) Field of the Invention

The invention relates to the technical field of organic wastewater treatment, and more particularly to a high efficiency biometric device for producing hydrogen and methane, in which continuous anaerobic fermentation reaction procedures, such as acidification, neutralization, methanation, separation and the like, are performed on the organic substance wastewater so that the sludge amount produced after treating the organic wastewater is decreased, the treated water with a good property is obtained, and the hydrogen and methane recovery are increased.

(2) Description of the Prior Art

When the organic substance in an anaerobic environment at the predetermined temperature, humidity and pH value encounters the microbe's anaerobic fermentation, the biogas is produced. The main components of the biogas include 50 to 80% of methane (CH₄), 20 to 50% of carbon dioxide (CO₂), and a little gas, such as carbon monoxide (CO), hydrogen sulfide (H₂S), hydrogen (H₂), oxygen (O₂) and nitrogen (N₂). Because the high concentrations of flammable gas of methane (CH₄) is present, the biogas may serve as the fuel. The biogas is produced from the anaerobic fermentation process of the organic wastewater. The organic substance of the produced biogas comes from the kitchen garbage, manure, organic wastewater, sludge, agricultural waste or municipal solid waste (MSW). The treatment for producing the biogas energy after the anaerobic fermentation or anaerobic removal is referred to as methanation.

In a conventional organic wastewater treatment method, as shown in FIG. 1, a flow-type anaerobic fermentation organic wastewater treatment device mainly adopts the flow-type design for continuously feeding the organic wastewater into the fermentation tank T1. After a period of time, the mixed solution of the treated removal liquid and microbes is drained, and methane and carbon dioxide produced by the anaerobic fermentation reaction may be discharged and collected from the top surface. In this device, after the mixed solution of the removal liquid and the microbes are drained, the microbe concentration in the fermentation tank T1 is decreased, and the methane production efficiency is low.

FIG. 2 shows a reflow-type anaerobic fermentation organic wastewater treatment device, which mainly improves the above-mentioned method and utilizes a precipitator T3 to precipitate the microbe sludge that is to be drained together with the removal liquid. The, the precipitated liquid flows back to mix with the wastewater and enters the fermentation tank T2. In this device, by the precipitating and flowing back to the fermentation tank T2, the hydraulic retention time of the anaerobic microbes can be ensured so that is serves as the matrix of the anaerobic microbes and is further decomposed, thereby enhancing the removal rate and the sludge reduction rate, and increasing the methane recovery.

FIG. 3 shows a filter-type anaerobic fermentation organic wastewater treatment device, which mainly makes the wastewater enter the fermentation tank T4 from the bottom of the fermentation tank T4. After passing through a filtering layer T5, the wastewater rises from bottom to top and then flows out. In addition to filtering the suspended substances, the filtering layer T5 also has many microbe groups to rapidly and effectively achieve the treatment effect and produce the methane and carbon dioxide. This device mainly adopts the filtering layer T5 to reserve the anaerobic microbes below the filtering layer T5, thereby enhancing the hydraulic retention time of the anaerobic microbes.

Typically, the anaerobic fermentation process of producing the methane must encounter two main stages. The first stage is the acidification stage of the acid producing phase, in which the complicated organic substances in the wastewater are transformed by the acidogenic bacteria and the facultative bacteria into the volatile organic acid (e.g., acetic acid, propionic acid, butyric acid, alcohol or the like). The second stage is the methane fermentation stage of producing the methane, wherein the metabolic reaction using the methanogenic bacteria, produced in the acidification process, the acetic acid and the propionic acid is performed to produce methane and carbon dioxide, so that the sludge content in the removal liquid is decreased.

Among various factors of influencing the methane by way of anaerobic fermentation, the following features can be concluded.

First, the growth of the biogas microbes is slow. The anaerobic fermentation utilizes the fermentation bacteria to decompose the organic substance and sufficiently provide the nutrient for the growth of the fermentation anaerobic bacteria, so that the biometric sludge concentration in the fermentation tank can be increased to accelerate the fermentation, and compensate for the drawback of the slow growth of the methanation bacteria. Thus, the reflowing or filtering treatment of the sludge is to lengthen the time when the sludge stays in the fermentation tank.

Second, the longer treating time is needed. The methanation process must pass the first stage acidification phase, the microbe's hydraulic retention time (HRT) is about 0.5 to 2 days so that the hydrogen and the carbon dioxide can be produced. The removal liquid is the volatile organic acid, such as acetic acid, propionic acid, butyric acid, alcohol or the like. The second stage methane fermentation phase is about 2 to 7 days (HRT), and the microbe's hydraulic retention time (HRT) is about 2 to 7 days, so that the acetic acid and the propionic acid have the metabolic reaction to produce the methane and carbon dioxide. The time of using the single-tank to treat the first stage acidification phase and the second stage methane fermentation phase is longer, and the continuous treatment cannot be performed.

Third, the concentration of the organic substance is too high. Because the pH value of the volatile organic acid removal liquid, such as acetic acid, propionic acid, butyric acid, alcohol or the like, produced in the first stage acidification phase, ranges from 5.0 to 6.5, and the preferred concentration pH value in the methane fermentation phase ranges from 7.2 to 7.6, the treatment efficiency of treating the first stage acidification phase and the second stage methane fermentation phase to produce the methane is decreased.

Fourth, the nitrogen concentration of the organic wastewater is too high. The concentrated waste also tends to affect the efficiency of anaerobic fermentation. For example, in the anaerobic fermentation treatment process of the pig's dung and urine, the pig's dung and urine have to be diluted with three times of water, so that the concentration of the ammonia nitrogen is lower than 1,500 ppm. Typically, the concentration of the ammonia nitrogen ranges from 1,500 to 3,000 ppm, which functions to inhibit the anaerobic bacteria. The concentration over 3,000 ppm produces poison.

At present, in the organic anaerobic wastewater treatment process, the middle temperature fermentation tank, the high-temperature fermentation tank and the device for speeding up the decomposition of the organic substance by adding the nutrient have been used, the technology has been well developed, and the commercial operation has been performed.

### SUMMARY OF THE INVENTION

In the methane fermentation procedure, the acidification phase and the methane fermentation phase have to be passed. So, the treatment device adopting the conventional single-tank fermentation design or dual-tank reflow design in FIGS. 1 to 3 is disadvantageous to the metabolic reaction of the acetic acid and propionic acid in the second stage methane fermentation phase, so that the efficiency of producing the methane cannot be increased. Therefore, the existing methane fermentation procedure is properly designed, the operations thereof are modified, and the front and rear fermentation tanks are designed according to the following reasons.

First, because microbe's hydraulic retention time (HRT) in the acidification phase is about 0.5 to 2 days, the microbe's hydraulic retention time (HRT) in the methane fermentation phase is about 2 to 7 days, so that the time of using the single-tank to treat the first stage acidification phase and the second stage methane fermentation phase is longer. Thus, in order to provide the continuous treatment, the front and rear fermentation tanks are adopted, and the rear tank preferably have multiple tanks, such as 1 tank to 3 tanks, 1 tank to 4 tanks or 1 tank to 5 tanks. This is a feasible and effective method.

Second, the pH value of the organic acid produced in the acidification phase ranges from 5.0 to 6.5, and the preferable pH value of the organic acid in the methane fermentation phase of producing the methane ranges from 7.2 to 7.6. So, when the rear tank is adopted, the pH value of the organic acid before entering the rear tank is neutralized, so that the pH value approximates to the preferred condition required by the methane fermentation phase. In this manner, the methanogenic bacteria can perform the sufficient metabolic reaction on the acetic acid and the propionic acid to produce the methane and carbon dioxide, the sludge can be decreased, ad the methane recovery can be increased.

It is therefore an object of the invention is to provide the design having front and rear fermentation tanks and a neutralization tank. The invention mainly sequentially performs the continuous anaerobic fermentation reaction procedures, such as acidification, neutralization, methanation and separation, on the organic wastewater mixed solution, so that the sludge amount produced by treating the organic wastewater is decreased, the treated water with a good property is obtained, and the hydrogen and methane recovery are increased.

The invention solves the above-identified technological problem and adopts the technological means by providing a high efficiency biometric device for producing hydrogen and methane. The device comprises: a mixing tank for collecting and accommodating an organic wastewater mixed solution; a two-stage anaerobic fermentation device for transforming the mixed solution, coming from the mixing tank, into hydrogen, methane, carbon dioxide and a removal liquid; and a solid liquid separation tank for performing filtering and separating on the removal liquid, coming from the two-stage anaerobic fermentation device. The characteristic lies in that: the two-stage anaerobic fermentation device comprises: a first anaerobic fermentation tank, which is connected to the mixing tank through a first conveying pipe, and for transforming the mixed solution, coming from coming from the mixing tank, into a first gas and a first liquid; a neutralization tank, which his connected to the first anaerobic fermentation tank through a second conveying pipe and for performing acid-base neutralization on the first liquid generated from the first anaerobic fermentation tank; a feeding tank, which is for accommodating an alkaline liquid, is connected to the neutralization tank through a third conveying pipe, and is for feeding alkaline liquid into the neutralization tank to make the first liquid approach neutral; and a second anaerobic fermentation tank composed of anaerobic fermentation tanks disposed in parallel. The second anaerobic fermentation tank is connected to the neutralization tank through a fourth conveying pipe, and is for transforming the first liquid, coming from the neutralization tank, into a second gas and a second liquid, and the second liquid is fed to the solid liquid separation tank through a fifth conveying pipe to perform filtering and separating. Thus, the removal rate (i.e., the sludge reduction rate) is increased, and the methane recovery is increased.

The first gas comprises hydrogen (H₂) and carbon dioxide (CO₂). The first liquid comprises volatile organic acid, such as acetic acid, propionic acid, butyric acid, alcohol or the like. The second gas comprises methane (CH₄) and carbon dioxide (CO₂). The second liquid comprises a biometric sludge removal liquid produced after the methanogenic bacteria performs the metabolic reaction on the acetic acid and the propionic acid.

In order to provide the better acidification reaction for the mixed solution in the first anaerobic fermentation tank, the mixing tank of the invention comprises a feeding port for providing the organic wastewater and a nutrient; a stirring unit for providing a uniform action on the mixed solution; a dilute water inlet for controlling a nitrogen concentration of the organic wastewater; and a cleaning drain port for cleaning the mixing tank.

The main technology of the invention adopts the first anaerobic fermentation tank and the second anaerobic fermentation tank arranged in the two-stage manner in conjunction with the acid-base neutralization tank, wherein the second anaerobic fermentation tank is composed of anaerobic fermentation tanks disposed in parallel, so that the invention has the following advantages in treating the organic wastewater: the produced biometric sludge amount is small; the useful energy of hydrogen (H₂), methane (CH₄) and carbon dioxide (CO₂) can be recycled; the one-to-many front and rear tank design can withstand the higher organic wastewater loading; the treated wastewater stability is high; no oxygen has to be provided; the parasite eggs can be killed; and the pathogens can be killed or inhibited.

In order to provide the better anaerobic fermentation reaction and produce the useful energy of hydrogen (H₂) methane (CH₄), carbon dioxide (CO₂) and the like, one of the first anaerobic fermentation tank and the second anaerobic fermentation tank of the invention may be selected from one of an anaerobic filter bed, a hybrid anaerobic filter bed, a baffled reactor and an up-anaerobic sludge bed (UASB), and is preferably the up-anaerobic sludge bed (UASB).

In the anaerobic fermentation process, the anaerobe tends to flow out when the liquid flows out of the fermentation tank. This phenomenon is referred to as the wash out phenomenon so that the anaerobe concentration is decreased, thereby decreasing the efficiency of producing the hydrogen and methane. Thus, a first liquid reflow tube is disposed on the first anaerobic fermentation tank of the invention, and second liquid reflow tubes connected to the anaerobic fermentation tanks are disposed on the second anaerobic fermentation tank, wherein the second liquid reflow tubes may be disposed independently or in parallel, and are preferably disposed in parallel.

In order to provide the methane fermentation reaction in the second anaerobic fermentation tank, the invention adopts a neutralization tank between the first anaerobic fermentation tank and the second anaerobic fermentation tank to perform acid-base neutralization on the volatile organic acid, and a feeding tank for providing an alkaline liquid, wherein stirring units are disposed in the neutralization tank.

In order to provide the filtering of the biometric sludge removal liquid in the anaerobic fermentation tanks and the treated water with a good property, the solid liquid separation tank adopted in the invention is a membrane bioreactor (MBR), which may be one of a branch type and an immersed type MBR, and is preferably the immersed type MBR.

In the branch type MBR, the active sludge is pumped to the tubular or flat sheet module at the high flow rate (usually higher than 2 m/s, and sometimes higher than 4 m/s), and the relatively large pressure drop and the extremely high transmembrane are generated. Because the operation quality is determined according to the transversal flow rate, the higher energy consumption is caused and the poor influence on the sludge is caused in order to obtain the larger flux. In addition, because the film surface area provided is smaller, the investment cost and the operation cost are higher.

In the immersed type MBR, the hollow fiber or flat sheet module is immersed into the aeration tank so that the treated water passes through the film by way of vacuuming. Because the film surface area is larger, the smaller flux can achieve the required flow, so that the energy consumption is lower and the scaling problem is less serious. Thus, the immersed type MBR is preferred in this invention.

In order to collect the hydrogen (H₂), methane (CH₄) and carbon dioxide (CO₂), a first gas collection tube is disposed on a top surface of the first anaerobic fermentation tank in this invention; and second gas collection tubes are disposed on top surfaces of the anaerobic fermentation tanks in the second anaerobic fermentation tank.

In order to provide the combustion for the first gas, collected by the first gas collection tube, and the second gas, collected by the second gas collection tube, the invention further comprises a generator.

In order to control the device operating and monitoring of the invention, the invention further comprises a control device, wherein the control device comprises a controller and a cable coil.

In addition to the application of the fixed type wastewater treatment, the invention may also be disposed on a mobile carrier to provide the non-fixed type wastewater treatment, wherein the mobile carrier is a container truck.

Further aspects, objects, and desirable features of the invention will be better understood from the detailed description and drawings that follow in which various embodiments of the disclosed invention are illustrated by way of examples.

### BRIEF DESCRIPTION OF THE DRAWINGS

FIG. 1 is a schematic view showing a conventional flow-type anaerobic fermentation organic wastewater treatment device.

FIG. 2 is a schematic view showing a conventional reflow-type anaerobic fermentation organic wastewater treatment device.

FIG. 3 is a schematic view showing a conventional filter-type anaerobic fermentation organic waste liquid treatment device.

FIG. 4 is a schematic view showing the device flow of the invention.

FIG. 5 is a schematic view showing a device system of the invention.

FIG. 6 is a pictorially schematic view showing the invention.

FIG. 7 is a pictorially schematic view showing the invention at another angle.

FIG. 8 is a schematic view showing the system of the invention connected to a generator and a control device.

FIG. 9 is a pictorially schematic view showing the invention disposed on a mobile vehicle.

FIG. 10 is a side perspective schematic view showing the invention disposed on the mobile vehicle.

FIG. 11 is a top perspective schematic view showing the invention disposed on the mobile vehicle.

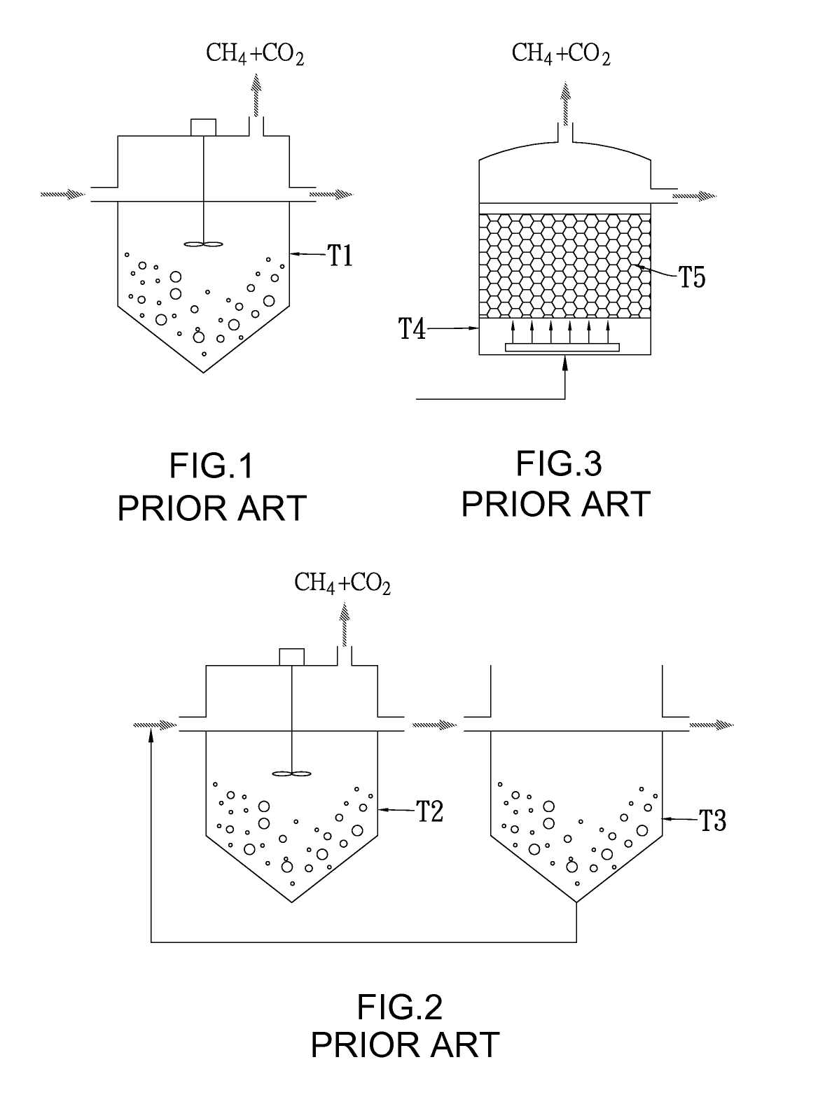

### DETAILED DESCRIPTION OF THE PREFERRED EMBODIMENTS

The above and other aspects of the invention will become better understood with regard to the following detailed description of the preferred but non-limiting embodiment. The following description is made with reference to the accompanying drawings. In addition to the fixed type wastewater treatment, the invention may also be disposed on a mobile container vehicle carrier to provide the non-fixed type wastewater treatment. In addition, in the following embodiment, the first gas comprises hydrogen (H₂) and carbon dioxide (CO₂); the first liquid is a volatile organic acid, such as acetic acid, propionic acid, butyric acid, alcohol or the like; the second gas comprises methane (CH₄), carbon dioxide (CO₂), carbon monoxide (CO), hydrogen sulfide (H₂S), hydrogen (H₂), oxygen (O₂) and nitrogen (N₂); and the second liquid is a biometric sludge removal liquid.

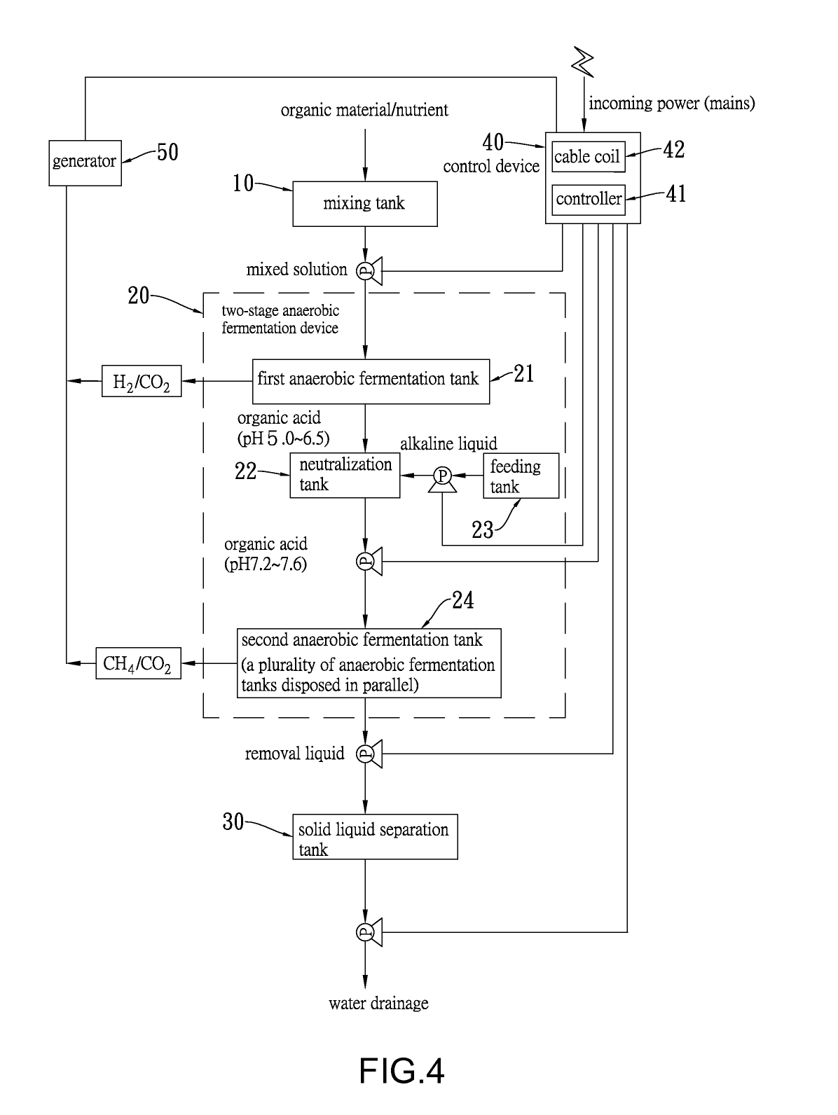

As show in FIG. 4, the optimum high efficiency biometric device for producing hydrogen and methane according to the invention usually comprises a mixing tank 10, a two-stage anaerobic fermentation device 20, a solid liquid separation tank 30, a control device 40 and a generator 50. The two-stage anaerobic fermentation device 20 comprises a first anaerobic fermentation tank 21, a neutralization tank 22, a feeding tank 23 and a second anaerobic fermentation tank 24. The control device 40 comprises a controller 41 and a cable coil 42.

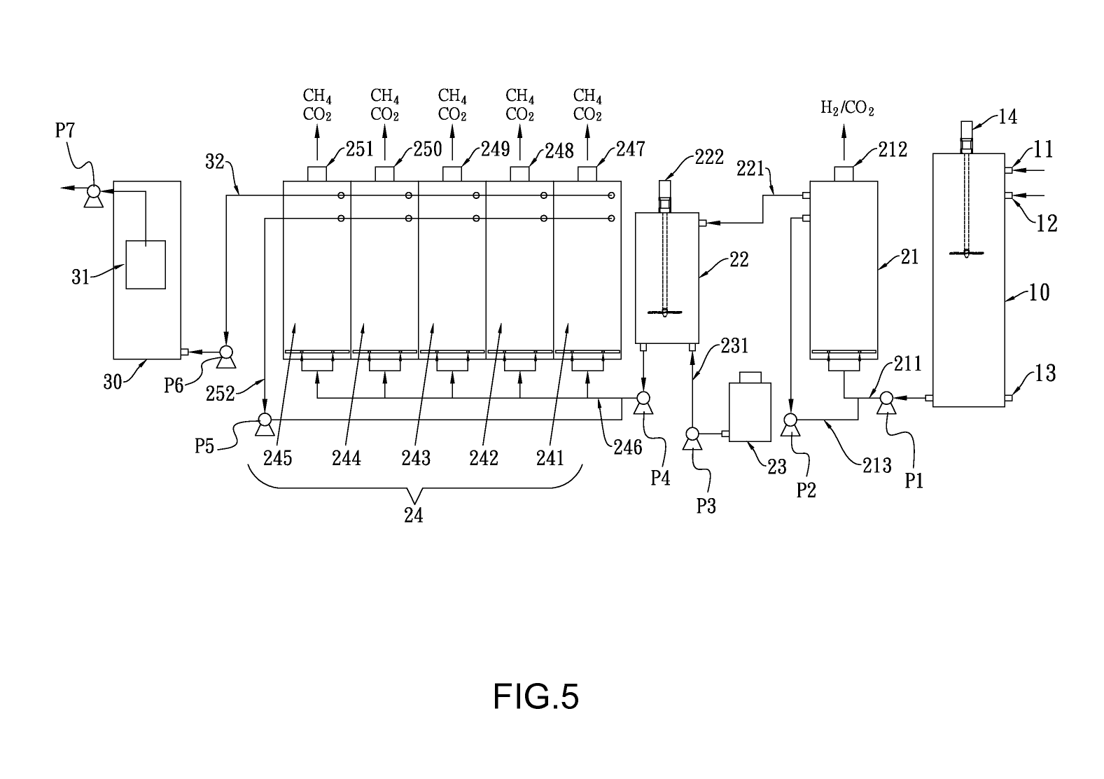

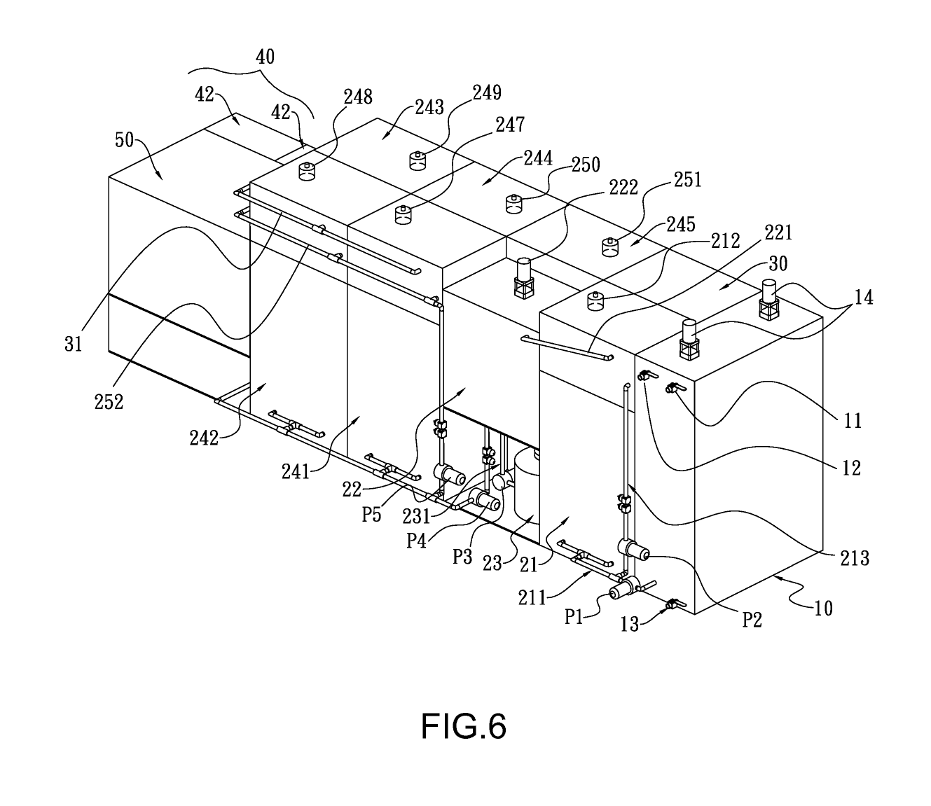

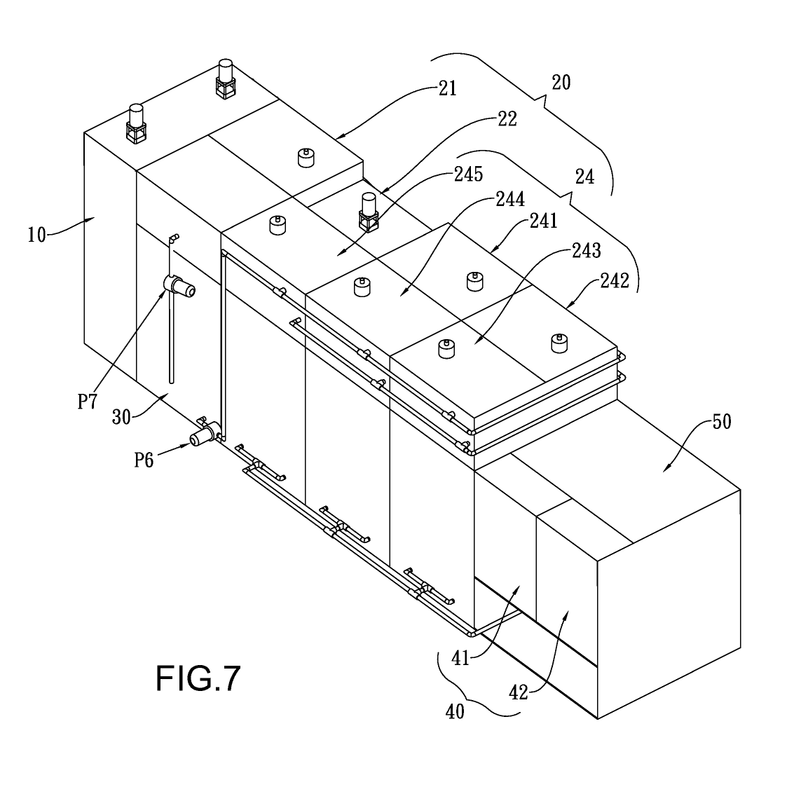

As shown in FIGS. 5 to 7, on the tank body of the mixing tank 10 are provided with: a feeding port 11 for collecting and accommodating the organic wastewater mixed solution; a dilute water inlet 12 for diluting the organic wastewater nitrogen concentration; a cleaning drain port 13 for cleaning the mixing tank 10; and two stirring units 14 disposed in the mixing tank 10 to provide the stirring actions.

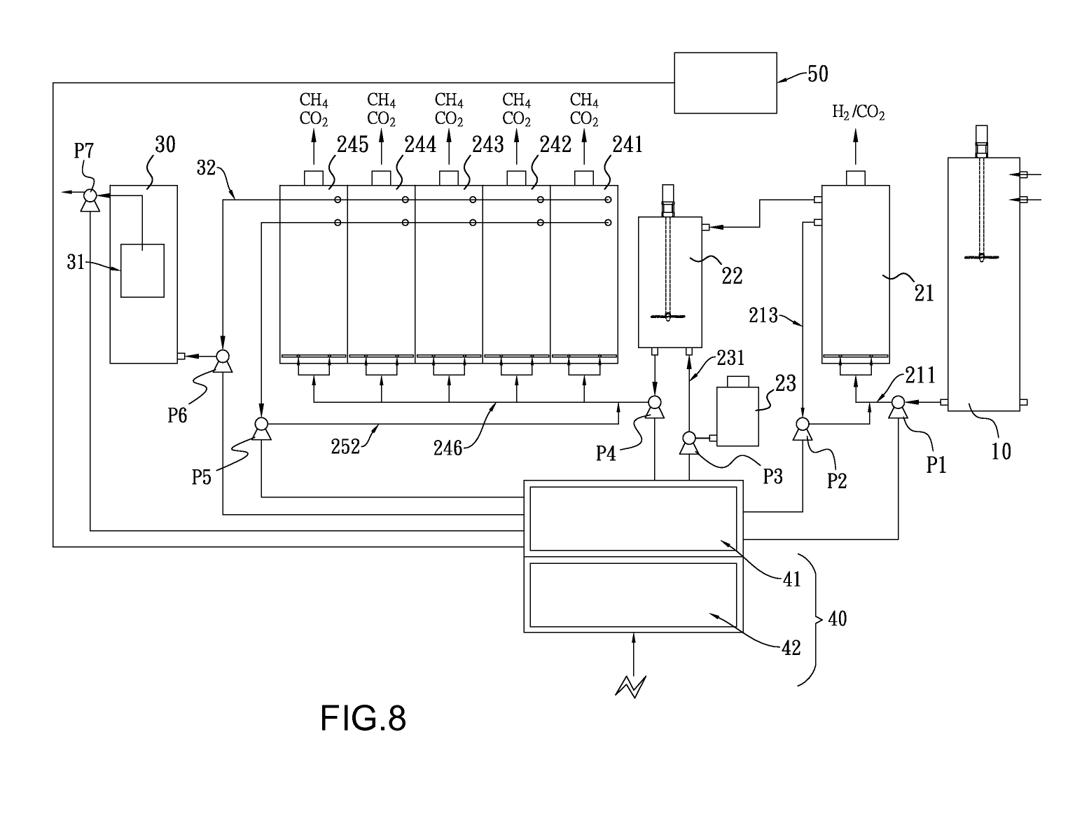

The first anaerobic fermentation tank 21 is an up-anaerobic sludge bed (UASB) for transforming the mixed solution, coming from the mixing tank 10, into a first gas and a first liquid. A first conveying pipe 211 below the first anaerobic fermentation tank 21 is connected to the mixing tank 10 to transfer the mixed solution from the mixing tank 10 to the first anaerobic fermentation tank 21. A first gas collection tube 212 for collecting the first gas is disposed on the top surface of the first anaerobic fermentation tank 21. A first liquid reflow tube 213 connected to the first conveying pipe 211 is disposed on the tank wall of the first anaerobic fermentation tank 21, and a pump P1 is disposed on the first conveying pipe 211. A pump P2 is disposed on the first liquid reflow tube 213. The pump P1 and the pump P2 are electrically connected to the controller 41 in the control device 40, as shown in FIG. 8. The pump P1 can transfer the mixed solution from the mixing tank 10 to the first anaerobic fermentation tank 21. The pump P2 can transfer the liquid from the first anaerobic fermentation tank 21 back to the first conveying pipe 211, and the liquid and the mixed solution in the mixing tank 10 again enter the first anaerobic fermentation tank 21.

A second conveying pipe 221, which is connected to the first anaerobic fermentation tank 21 and for performing acid-base neutralization on the first liquid coming from the first anaerobic fermentation tank 21, is disposed on the neutralization tank 22. In order to make the first liquid (floating liquid) naturally flow into the neutralization tank 22, the second conveying pipe 221 is disposed slantingly. In addition, a stirring unit 222 for providing stirring actions is disposed in the neutralization tank 22.

A third conveying pipe 231, which is connected to the neutralization tank 22 and is for feeding the alkaline liquid into the neutralization tank 22 to perform acid-base neutralization with the first liquid so that the pH value of the first liquid approximates to the neutral value, is disposed on the feeding tank 23. Also, a pump P3 electrically connected to the controller 41 of the control device 40 is disposed on the third conveying pipe 231. As shown in FIG. 8, the pump P3 can feed the alkaline liquid from the feeding tank 23 to the neutralization tank 22.

The second anaerobic fermentation tank 24 is composed of five anaerobic fermentation tanks 241 to 245 and for transforming the first liquid, coming from the neutralization tank 22, into the second gas and the second liquid, wherein each of anaerobic fermentation tanks 241 to 245 is connected to the neutralization tank 22 with a fourth conveying pipe 246. The bottoms of the anaerobic fermentation tanks 241 to 245 are connected to the fourth conveying pipe 246 in parallel, so that the first liquid in the neutralization tank 22 is uniformly fed into each of the anaerobic fermentation tanks 241 to 245. In addition, second gas collection tubes 247 to 251 for collecting the second gas are disposed on tops of the anaerobic fermentation tanks 241 to 245. A second liquid reflow tube 252 connected to the fourth conveying pipe 246 is disposed on the tank wall of each of the anaerobic fermentation tanks 241 to 245. Also, a pump P4 is disposed on the fourth conveying pipe 246, and a pump P5 is disposed on the second liquid reflow tube 252. The pump P4 and the pump P5 are electrically connected to the controller 41 of the control device 40. As shown in FIG. 8, the pump P4 can uniformly feed the first liquid from the neutralization tank 22 into each of the anaerobic fermentation tanks 241 to 245, and the pump P5 can feed the liquid from each of the anaerobic fermentation tanks 241 to 245 back to the fourth conveying pipe 246 and then into the anaerobic fermentation tanks 241 to 245.

The solid liquid separation tank 30 is an immersed type membrane bioreactor (MBR), in which a filtering module 31 is disposed. A fifth conveying pipe 32 connected to the tops of the anaerobic fermentation tanks 241 to 245 in parallel is disposed on the bottom of the solid liquid separation tank 30, and for feeding the second liquid (floating liquid) from each of the anaerobic fermentation tanks 241 to 245 to the bottom of the solid liquid separation tank 30. The filtering module 31 separates the second liquid into the biometric sludge and the wastewater that can be drained. In addition, a pump P6 is disposed on the fifth conveying pipe 32, a pump P7 is disposed on the filtering module 31, and the pump P6 and the pump P7 are electrically connected to the controller 41 of the control device 40. As shown in FIG. 8, the pump P6 can feed the second liquid removal liquid from each of the anaerobic fermentation tanks 241 to 245 into the solid liquid separation tank 30, and the pump P7 can discharge the water, obtained after the second liquid (removal liquid) in the solid liquid separation tank 30 is filtered and separated.

As shown in FIGS. 4, 5 and 8, the invention further has a generator 50 and a control device 40. The generator 50 generates the power after by combusting the collected first gas and second gas.

As shown in FIG. 8, the control device 40 comprises the controller 41 and the cable coil 42, wherein the cable coil 42 can be connected to the incoming power, and the controller 41 is electrically connected to the pumps P1, P2, P3, P4, P5, P6 and P7 and for operating and monitoring the devices of invention, so that the mixing tank 10, the first anaerobic fermentation tank 21, the neutralization tank 22, the feeding tank 23, the second anaerobic fermentation tank 24 and the solid liquid separation tank 30 can continuously operate and treat the organic wastewater.

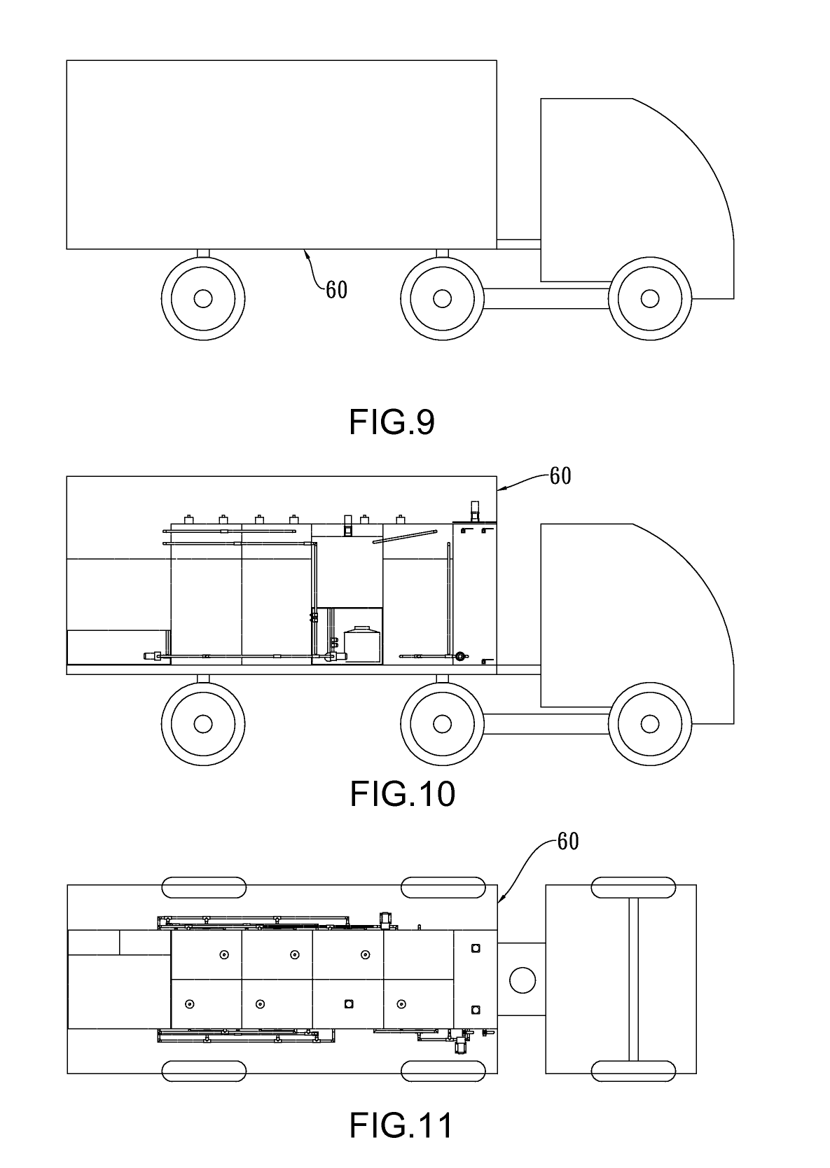

As shown in FIGS. 9 to 11, the high efficiency biometric device for producing hydrogen and methane according to the invention is disposed in a container truck 60 to further provide the non-fixed type organic wastewater treatment. FIG. 9 is a pictorially schematic view showing the invention disposed on a mobile vehicle. FIG. 10 is a side perspective schematic view showing the invention disposed on the mobile vehicle. FIG. 11 is a top perspective schematic view showing the invention disposed on the mobile vehicle.

In summary, the high efficiency biometric device for producing hydrogen and methane is designed according to the above-mentioned optimum conditions and can indeed increase the removal rate and the sludge reduction rate, and increase the methane recovery. In addition, the generator for combusting the recycled gas is provided so that the invention also advantageously has the low energy consumption, can decrease organic waste contamination, and also increase the green energy production pathway.

New characteristics and advantages of the invention covered by this document have been set forth in the foregoing description. It is to be expressly understood, however, that the drawings are for the purpose of illustration only and are not intended as a definition of the limits of the invention. Changes in methods, shapes, structures or devices may be made in details without exceeding the scope of the invention by those who are skilled in the art. The scope of the invention is, of course, defined in the language in which the appended claims are expressed.

What is claimed is:

1. A high efficiency biometric device for producing hydrogen and methane, comprising:
a mixing tank for collecting and accommodating an organic wastewater mixed solution;
a two-stage anaerobic fermentation device for transforming the mixed solution in the mixing tank into hydrogen, methane, carbon dioxide and a removal liquid; and
a solid liquid separation tank for performing filtering and separating on the removal liquid, coming from the two-stage anaerobic fermentation device, to decrease sludge and obtain treated water with a good property;
wherein the characteristic lies in that the two-stage anaerobic fermentation device comprises:
a first anaerobic fermentation tank, which is connected to the mixing tank through a first conveying pipe, and for transforming the mixed solution, coming from coming from the mixing tank, into a first gas and a first liquid;
a neutralization tank, which his connected to the first anaerobic fermentation tank through a second conveying pipe and for performing acid-base neutralization on the first liquid generated from the first anaerobic fermentation tank;
a feeding tank, which is for accommodating an alkaline liquid, is connected to the neutralization tank through a third conveying pipe, and is for feeding alkaline liquid into the neutralization tank to make the first liquid approach neutral; and
a second anaerobic fermentation tank composed of anaerobic fermentation tanks disposed in parallel, wherein the second anaerobic fermentation tank is connected to the neutralization tank through a fourth conveying pipe, and is for transforming the first liquid, coming from the neutralization tank, into a second gas and a second liquid, and the second liquid is fed to the solid liquid separation tank through a fifth conveying pipe to perform filtering and separating.

2. The high efficiency biometric device according to claim 1, wherein the first anaerobic fermentation tank is one of an anaerobic filter bed, a hybrid anaerobic filter bed, a baffled reactor and an up-anaerobic sludge bed (UASB).

3. The high efficiency biometric device according to claim 1, wherein a first gas collection tube is disposed on a top surface of the first anaerobic fermentation tank.

4. The high efficiency biometric device according to claim 1, wherein each of the anaerobic fermentation tanks is one of an anaerobic filter bed, a hybrid anaerobic filter bed, a baffled reactor and an up-anaerobic sludge bed (UASB).

5. The high efficiency biometric device according to claim 1, wherein a second gas collection tube is disposed on a top surface of the anaerobic fermentation tanks.

6. The high efficiency biometric device according to claim 1, further comprising a generator for generating power by combusting the collected first gas and second gas.

7. The high efficiency biometric device according to claim 6, further comprising a control device for controlling operations of the biometric device and the generator, wherein the control device comprises a controller and a cable coil.

8. The high efficiency biometric device according to claim 1, wherein the solid liquid separation tank is one of branch type and immersed type membrane bioreactors.

9. The high efficiency biometric device according to claim 1, wherein the neutralization tank further comprises a stirring unit.

10. The high efficiency biometric device according to claim 1, further comprising a mobile carrier.

11. The high efficiency biometric device according to claim 10, wherein the mobile carrier is a container truck.
